(19) United States
(12) Patent Application Publication
(10) Pub. No.: US 2012/0221083 A1
(43) Pub. Date: Aug. 30, 2012

(54) Treatment System by Heat Extraction and Methods of Use Thereof

(76) Inventor: Meliza Cruzada, Lakeside, CA (US)

(21) Appl. No.: 13/406,468

(22) Filed: Feb. 27, 2012

Related U.S. Application Data
(60) Provisional application No. 61/447,082, filed on Feb. 27, 2011.

(51) Int. Cl. A61F 7/10

(52) U.S. Cl. 607/112

(57) ABSTRACT

A treatment system utilizing cryolipolysis to reduce subcutaneous lipid-rich areas by removing heat from the subcutaneous lipid-rich area, thereby affecting lipid-rich cells of the subcutaneous lipid-rich areas having an applicator having a cavity, and a retention mechanism, and a cooling member, where the cavity contains the cooling member, and the retention mechanism secures the applicator to a target area.

### CROSS-REFERENCE TO RELATED APPLICATION

This application claims the benefit of priority to U.S. Provisional Application No. 61/447,082 filed on Feb. 27, 2011, which is incorporated herein by reference in its entirety.

### BACKGROUND

Excess body fat, or adipose tissue, can detract from personal appearance and athletic performance, and can pose significant health risks by increasing the likelihood of developing various types of diseases, for example, heart disease, high blood pressure, osteoarthritis, bronchitis, hypertension, diabetes, deep-vein thrombosis, pulmonary emboli, varicose veins, gallstones, and hernias.

Surgical procedures such as liposuction have been employed to remove excess body fat. Due to the invasive and cost prohibitive nature of such surgical procedures, alternative non-invasive body contouring procedures have become a common and growing demand. Many non-invasive body contouring procedures exist to remove or reduce adipose cells including topical agents, massages, acupuncture, weight-loss drugs, exercise, dieting, and applying heat to subcutaneous lipid-rich areas. However, each of the methods have limitations making the methods ineffective or impractical in certain circumstances.

Studies have shown that cooling subcutaneous lipid-rich areas results in crystallization of cytoplasmic lipid deposits within adipose cells resulting in cell damage or cell death. Immune cells engulf the affected adipose cells and eliminate them from the body. The remaining fat layer condenses, reducing fat volume at the target area.

### SUMMARY OF THE INVENTION

The present disclosure pertains to a treatment system for extracting heat from subcutaneous lipid-rich areas, having an applicator having a cavity and a retention mechanism, and a cooling member, where the cavity contains the cooling member, and the retention mechanism secures the applicator to a target area. One aspect of the disclosure is a system is configured to reduce the temperature of a target area such that the subcutaneous lipid-rich area is affected.

Another aspect of the disclosure is a system where the cavity slidably engages the retention mechanism. Another aspect of the disclosure is a system where the retention mechanism comprises a strap, plate, aperture, fastening mechanism, and loop. Another aspect of the disclosure is a system where the fastening mechanism secures the treatment system to a location on the user's body. Another aspect of the disclosure is a system where the fastening mechanism is adjustable to increase or decrease the pressure applied to a target area. Another aspect of the disclosure is a system where the fastening mechanism applies pressure to the target area that is approximately equal to or greater than the systolic blood pressure in the user's non-lipid-rich area of the target area. Another aspect of the disclosure is a system where the fastening mechanism is adjustable to allow for the system to be used on a plurality of locations on the user's body.

Another aspect of the disclosure is a system having an opening where the cooling member passes through the opening. Another aspect of the disclosure is a system where the applicator comprises a flexible section for allowing the circumference of the loop to be altered. Another aspect of the disclosure is a system where the applicator comprises a closure mechanism. Another aspect of the disclosure is a system where the applicator comprises an opening device. Another aspect of the disclosure is a system where the applicator comprises a temperature control mechanism. Another aspect of the disclosure is a system where the temperature control mechanism is a cryoprotectant. Another aspect of the disclosure is a system where the cooling member has an acuate shape for forming to the contour o the target area. Another aspect of the disclosure is a system where the cooling member is cooled.

Another aspect of the disclosure is a method for affecting lipid-rich cells having the steps of applying a treatment system to a target area to affect a subcutaneous lipid-rich area, where the treatment system has a cooling member and an applicator, and the applicator has a cavity and retention mechanism.

Another aspect of the disclosure is a method where the cooling member is maintained at an average temperature between about −20 and 10° C. Another aspect of the disclosure is a method where the cooling member is maintained at an average temperature between about −20 and 5° C. Another aspect of the disclosure is a method where the cooling member is maintained at an average temperature between about −20 and 0° C. Another aspect of the disclosure is a method where the cooling member is maintained at an average temperature between about −20 and −5° C. Another aspect of the disclosure is a method where the cooling member is maintained at an average temperature between about −20 and −10° C. Another aspect of the disclosure is a method where the cooling member is maintained at an average temperature between about −20 and −15° C.

Another aspect of the disclosure is a method where the lipid-rich cells are cooled to an average temperature between about −10 and 10° C. Another aspect of the disclosure is a method where the lipid-rich cells are cooled to an average temperature between about −10 and 5° C. Another aspect of the disclosure is a method where the lipid-rich cells are cooled to an average temperature between about −10 and 0° C. Another aspect of the disclosure is a method where the lipid-rich cells are cooled to an average temperature between about −10 and −5° C.

Another aspect of the disclosure is a method where the lipid-rich cells are cooled to an average first temperature range for a first period of time and cooled to an average second temperature range for a second period of time. Another aspect of the disclosure is a method where the average first temperature range is between about −5 and 0° C. Another aspect of the disclosure is a method where the average second temperature range is between about 0 and 5° C.

With those and other objects, advantages and features on the invention that may become hereinafter apparent, the nature of the invention may be more clearly understood by reference to the following detailed description of the invention, the appended claims, and the drawings attached hereto.

### BRIEF DESCRIPTION OF THE DRAWINGS

The accompanying drawings, which are incorporated herein and form part of the specification, illustrate various embodiments of the present invention and together with the description, further serve to explain the principles of the invention and to enable a person skilled in the pertinent art to make and use the invention. In the drawings, like reference numbers indicate identical or functionally similar elements. A more complete appreciation of the invention and many of the attendant advantages thereof will be readily obtained as the same becomes better understood by reference to the following detailed description when considered in connection with the accompanying drawings, wherein:

FIG. 1 is a perspective view of a treatment system according to an exemplary embodiment.

FIG. 2 is a side view of a treatment system according to an exemplary embodiment.

FIG. 3 is a side view of a treatment system according to an exemplary embodiment.

FIG. 4 is a plan view of a treatment system according to an exemplary embodiment.

FIG. 5 is a perspective view of a treatment system according to an exemplary embodiment.

FIG. 6 is a perspective view of a treatment system according to an exemplary embodiment.

FIG. 7 is a perspective view of a portion of a treatment system according to an exemplary embodiment.

### DETAILED DESCRIPTION

In the following detailed description, reference is made to the accompanying drawings which form a part hereof and in which is shown by way of illustration specific embodiments in which the invention may be practiced. These embodiments are described in sufficient detail to enable those skilled in the art to practice the invention, and it is to be understood that other embodiments may be utilized and that structural or logical changes may be made without departing from the scope of the present invention. The following detailed description is, therefore, not to be taken in a limiting sense, and the scope of the present invention is defined by the appended claims.

The term “user,” as used herein, includes any animal having blood, adipose tissue, and an epidermis. In preferred embodiments, the subject is a human or some other mammal and even more preferable a non-infant human.

The term “affecting,” as used herein, includes affecting, disrupting, shrinking, disabling, destroying, removing, killing, or the like.

The term “target area,” as used herein, includes the surface area on a location of the user's body that contacts the applicator 200 and associated subsurface area, and includes the non-lipid-rich areas such as the dermis and epidermis, and the subcutaneous lipid-rich areas.

The term “subcutaneous lipid-rich area,” as used herein, includes areas where tissue lies beneath the dermis and includes adipose tissue that may be composed primarily of lipid-rich cells, or adipocytes.

The present disclosure pertains to a treatment system 100 for extracting heat from subcutaneous lipid-rich areas. The treatment system 100 is a non-invasive system utilizing cryolipolysis to reduce subcutaneous lipid-rich areas by removing heat from the subcutaneous lipid-rich area, thereby affecting lipid-rich cells of the subcutaneous lipid-rich areas.

The cooling of subcutaneous lipid-rich areas to a temperature lower than 37° C. specifically targets subcutaneous lipid-rich cells. Non-lipid-rich cells of the epidermis, dermis and surrounding tissue are more resilient to colder temperatures when compared to that of lipid-rich cells. Thus, the removal of heat from the subcutaneous lipid-rich area affects the lipid-rich cells without affecting the non-lipid-rich cells of the dermis, epidermis and other surrounding tissue.

Actions affecting the subcutaneous lipid-rich area are believed to result from at least one mechanism acting alone or in combination. For example, it is thought that such mechanism or mechanisms trigger an apoptotic cascade that causes the dominant form of lipid-rich cell death by non-invasive cooling. Apoptosis, also referred to as “programmed cell death”, is a genetically-induced death mechanism by which cells self-destruct without incurring damage to surrounding tissues. Extracting heat from cells utilizing cold exposure is one mechanism that can induce cellular apoptosis.

Apoptotic cells display phagocytic markers on the surface of the cell membrane, thus marking the cells for phagocytosis by macrophages. Phagocytes remove the marked cells without commencing an immune response. Extracting heat from cells utilizing cold exposure in lipid-rich cells can results in permanent reduction and reshaping of subcutaneous lipid-rich areas.

In addition, extracting heat from cells utilizing cold exposure is believed to induce lipolysis, or fat metabolism, of lipid-rich cells and has been shown to enhance existing lipolysis thereby further affecting lipid-rich cells.

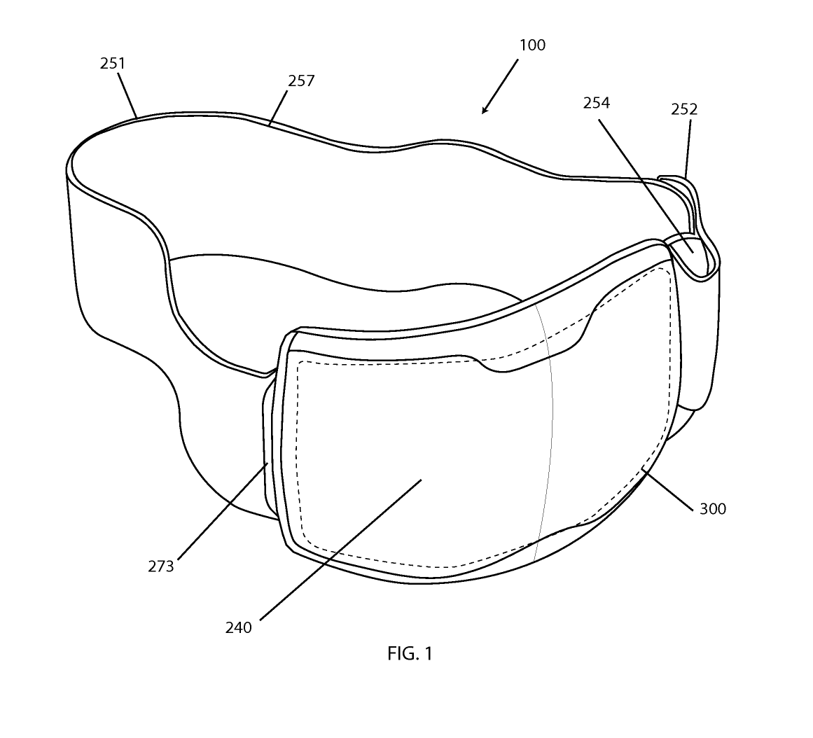

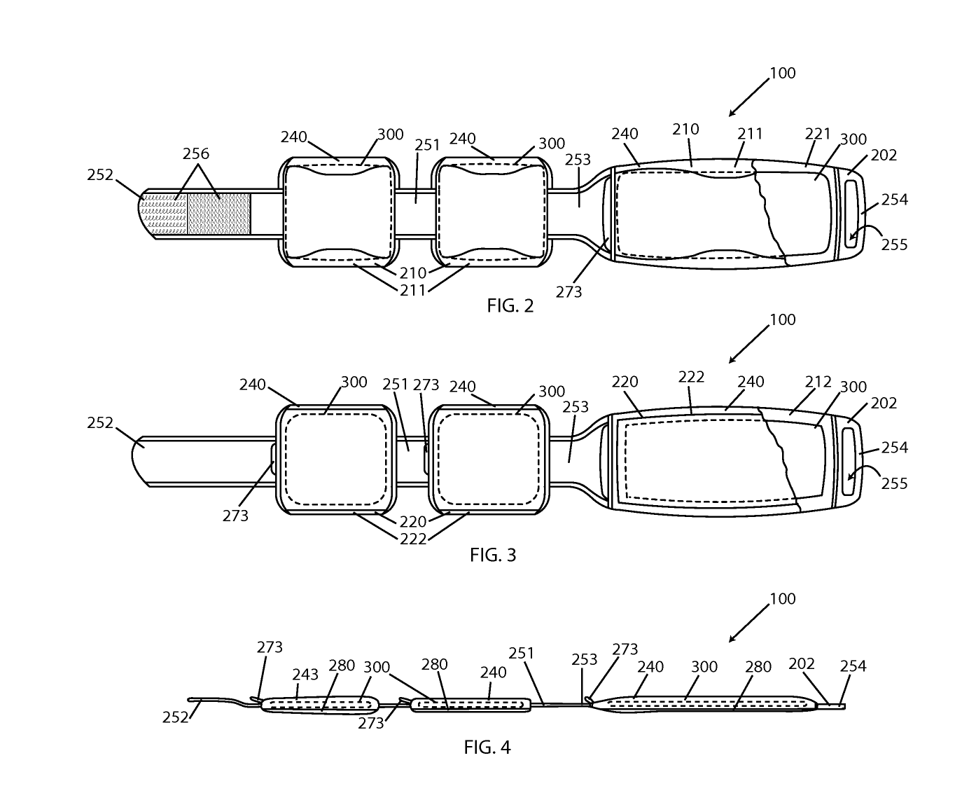

In one embodiment, as shown in FIG. 1-5, the treatment system 100 has a cooling member 300 and an applicator 200. The applicator device has end 202, at least one cavity 240, and a retention mechanism. The cavity 240 has front member 210, rear member 220 where the front member 210 engages the rear member 220 thereby creating a cavity 240 and an opening 230. The front member 210 has exterior surface 211 and interior surface 212. The rear member 220 has interior surface 221 and exterior surface 222. The cavity 240 receives the cooling member 300 by way of the opening 230. In one embodiment, the cavity 240 can engages the retention mechanism. In one embodiment, the cavity 240 slidably engages the retention mechanism thereby allowing for the cavity 240 to slide along the retention mechanism and be positioned at a desired location along the retention mechanism.

As shown in FIG. 5, the opening 230 allows for the cooling member 300 to be received by the cavity 240. While the opening 230 is preferably positioned in a vertical orientation on the side of the cavity 240, as shown in FIGS. 5 and 7, the opening 230 can be located at any orientation, including a horizontal orientation on the top of the cavity 240.

In one embodiment, by way of example, the exterior surface 222 contacts the target area for removing heat from, or cooling, the subcutaneous lipid-rich area of the user upon securing the applicator 200 to the user. The applicator 200 can be applied to all suitable locations on a user's body, for example, without limitation, abdominal area, waist area, neck area, arm area, shoulder area, buttocks area, thigh area, the like, or any combination thereof. The application device 200 can have various shapes and sizes suitable for placement at different locations on a user's body allowing for the removal of heat from any subcutaneous lipid-rich area of the user.

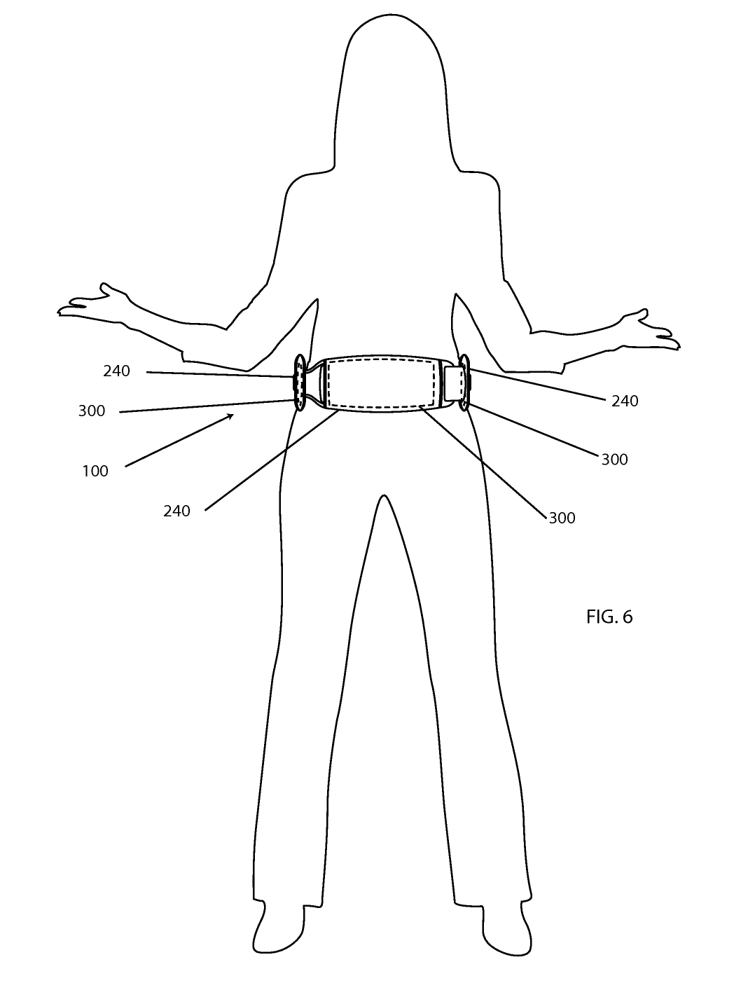

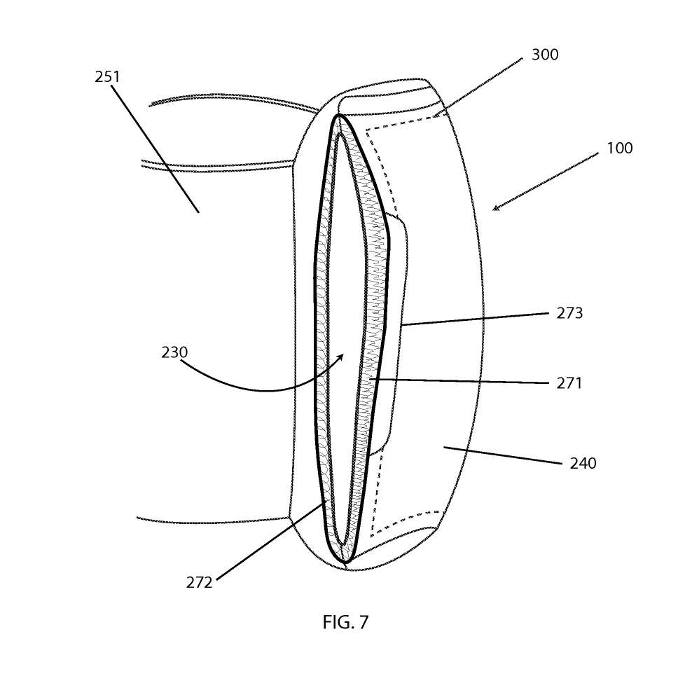

Applicator 200 can have any size, shape, or number of cavities 240 depending on the location, size, and number of target areas to be treated. For example, as shown in FIG. 6, where the target areas to be treated are the abdominal area, the left waist line area, and the right waist line area, the applicator 200 has three cavities 240 where each cavity 240 can be positioned on a desired target area, specifically where one cavity 240 is positioned on abdominal area, one cavity 240 is positioned on the left waist line, and one cavity 240 is positioned on the right waist line. Also by way of example, the cavities on the left waist line and right waist line are shaped and sized to cover the left waist line area and right waist line area, respectively, and the cavity on the abdominal area is shaped and sized to cover the abdominal area of a user's abdomen area.

The applicator 200 is composed of durable material, for example, without limitation, cotton, wool, linen, or manmade polymeric materials such as neoprene, spandex, polypropylene, polyester, nylon, polyethylene, metallocene catalyst polyethylene, or the like.

The retention mechanism is any mechanism for securing the applicator 200 to the user, thereby positioning the cavity 240 on the target area. In one embodiment, as shown in FIGS. 1-5, the retention mechanism has a strap 251 with end 252 and end 253, a plate 254 with an aperture 255 for receiving the strap 251, and a fastening mechanism 256 for securing the strap 251 in place thereby preventing the circumference of the loop 257 created by securing the strap 251 to the applicator 200 from increasing or decreasing in length. In one embodiment, the fastening mechanism 256 engages end 252 of the strap 251. While the fastening mechanism 256 is preferably hook and loop, the fastening mechanism 256 can be any mechanism for securing a strap 251 in place, for example, snap, buckle, clip, button, hook, male and female connector, or the like. In one embodiment, the aperture 255 can receive the strap 251 and fold back on itself thereby engaging the hook and loop of the strap 251, creating a loop 257 and securing the strap 251 into place.

In one embodiment, the retention mechanism is a hook and loop mechanism positioned on strap 251 and end 202 of the applicator 200. In one embodiment, the retention mechanism is a hook and spandex mechanism where the hook engages the spandex.

In one embodiment, the fastening mechanism 256 allows for adjusting the length or circumference of the loop 257. In one embodiment, the fastening mechanism 256 can adjust the length or circumference of the loop 257 thereby allowing for the applicator 200 to be used in relation to various locations on a user's body.

Applying pressure to the subcutaneous lipid-rich area can be advantageous to achieve efficient cooling of the lipid-rich cells. Heat extracted from the surface of the subcutaneous lipid-rich area creates a temperature gradient within the subcutaneous lipid-rich area which in turn cools first epidermis, dermis, and finally subcutaneous lipid-rich cells. Dermal blood flow brings heat from the body core to the dermis thereby limiting cooling of the deep dermis and subcutaneous lipid-rich cells. Reducing blood flow to the subcutaneous lipid-rich area can decrease the amount of heat brought from the body core to the dermis. Pressure to the subcutaneous lipid-rich area greater than the systolic blood pressure can reduce blood flow to the subcutaneous lipid-rich area. By decreasing the circumference of the applicator 200, an increased amount of pressure is applied to the subcutaneous lipid-rich area, thereby improving the efficiency of reducing the temperature of the subcutaneous lipid-rich area.

In one embodiment, the fastening mechanism 256 allows for the amount of pressure applied to the subcutaneous lipid-rich area to be increased or decreased. In one embodiment, the fastening mechanism 256 allows for the pressure to the subcutaneous lipid-rich area to be equal to or greater than the systolic blood pressure in the user's non-lipid-rich area of the target area so as to decrease the blood flow within this target area.

In one embodiment, the applicator 200 has a flexible section (not shown) that allows for the length or circumference of the loop 257 to be altered. As the treatment system 100 is utilized, lipid-rich cells will be affected thereby decreasing the circumference of the user's body at the location where the treatment system 100 is utilized. The flexible section maintains a tight fit around the user's body after the circumference of the user's body has decreased thereby allowing the user to continue using the treatment system 100 after the circumference of the user's body has decreased. While the flexible section can be positioned at any place along the length of the applicator 200, the flexible section is preferably positioned within the strap 251. In one embodiment, the flexible section is a section of the applicator 200 made of elastic that stretches when pulled or when the applicator 200 is wrapped around the user's body. In one embodiment, the applicator 200 is made of flexible material that allows for the length or circumference of the applicator 200 to be altered. For example, the applicator 200 can be made of neoprene, spandex, polypropylene, polyester, nylon, polyethylene, metallocene catalyst polyethylene, or the like

In one embodiment, as shown in FIG. 7, the applicator 200 has closure mechanism for closing opening 230. In one embodiment, by way of example, the closure mechanism has first closure member 271 engaged to interior surface 212 of front member 210 and second closure member 272 engages to interior surface 221 of rear member 220. The first closure member 271 engages the second closure member 272, thereby closing the opening 230. While the closure mechanism can be any mechanism for closing an opening 230, for example, a zipper, button, snap, buckle, clip, hook, or the like, the closure mechanism is preferably a hook and loop mechanism.

In one embodiment, the applicator 200 has an opening device 273 for opening the closure mechanism. For example, the opening device 273 can be a tab to aid the user in separating the first closure member 271 from the second closure member 272.

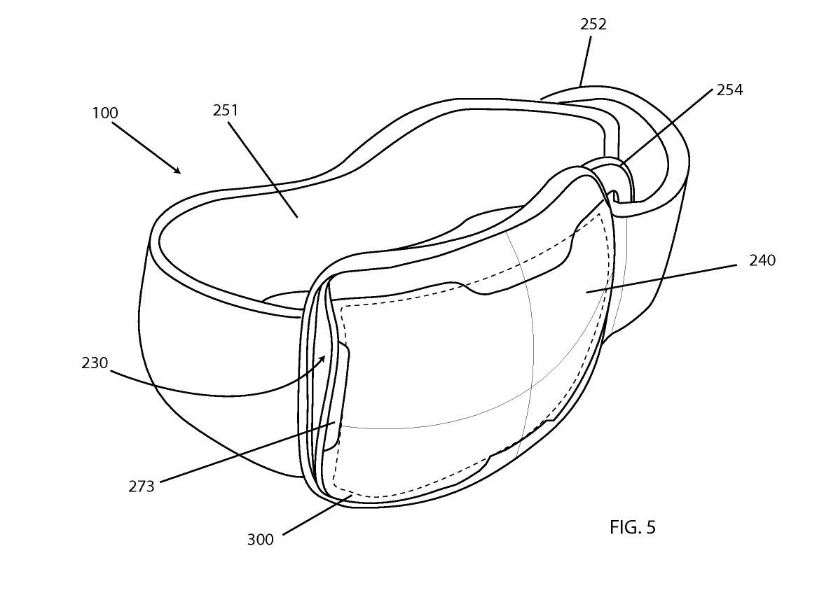

In one embodiment, as shown in FIG. 4, the applicator 200 has a temperature control mechanism 280 for controlling the temperature received by the subcutaneous lipid-rich area. In one embodiment, the temperature control mechanism 280 allows for heat to be extracted from the subcutaneous lipid-rich cells utilizing cold exposure while preventing the non-lipid-rich cells and tissues in the surrounding areas from freezing. In one embodiment, the temperature control mechanism 280 allows for the temperature received by the subcutaneous lipid-rich area to be from about −5 to 10° C. In one embodiment, the temperature control mechanism 280 is an increase in the thickness of the front member 210 and/or rear member 220, whereby an increase in the thickness of the front member 210 and/or rear member 220 decreases the amount of heat extracted from the subcutaneous lipid-rich area by the cooling member 300. In one embodiment, temperature control mechanism 280 is a layer of cryoprotectant. In one embodiment, the cryoprotectant is applied to the exterior surface 222 of rear member 220 or exterior surface 211 of front member 210.

The cooling member 300 can be any device that can be received by cavity 240 that extracts heat from subcutaneous lipid-rich areas. In one embodiment, the cooling member 300 is a quantity of gel beads or any other form fitting moldable gel pack that retains cold when cooled. The cooling member 300 can have water, alcohol, glycol, organic materials, inorganic materials, synthetic heat transfer fluid, oil, refrigerant, substances that can be pre-cooled and that extract heat over an extended period of time, the like, or any combination thereof. The cooling member 300 can have any shape and size that extracts heat from subcutaneous lipid-rich areas. In one embodiment, the cooling member 300 is substantially rectangular. In one embodiment, the cooling member 300 can have a thickness is the range about 0.6 cm to 5 cm. The cooling member 300 preferably has a thickness of 2 cm. In one embodiment, the cooling member 300 has an acuate shape for forming to the contour of the target area. The cooling member 300 can be frozen upon placement in a freezer at a temperature below freezing.

The following describes the method of use of the treatment system 100. The cooling member 300 is frozen. At least one cooling member 300 is passed through at least one opening 230 and into at least one cavity 240. At least one closure mechanism closes at least one opening 230. The applicator 200 is then wrapped around the user and positioned at a desired location on a user's body allowing for the cavity 240 to be positioned on a desired target area. The retention mechanism secures the applicator 200 to the user by passing the strap 251 through the aperture 255 and folding the strap 251 back on itself thereby engaging the hook with loop 257. The retention mechanism is adjusted to apply pressure to the target area. The applicator 200 remains on the target area for 60 minutes. The aforementioned steps are repeated every other day for 12 weeks and then repeated by positioning the applicator 200 on another location of a user's body.

In some embodiments, the cooling member 300 is maintained at an average temperature between about −20 and 10° C. In other embodiments, the cooling member 300 is maintained at an average temperature between about −20 and 5° C., at an average temperature between about −20 and 0° C., at an average temperature between about −20 and −5° C., at an average temperature between about −20 and −10° C., or at an average temperature between about −20 and −15° C.

In some embodiments, the lipid-rich cells are cooled to an average temperature between about −10 and 10° C. In other embodiments, the lipid-rich cells are cooled to an average temperature between about −10 and 5° C., to an average temperature between about −10 and 0° C., or to an average temperature between about −10 and −5° C.

In some embodiments, the lipid-rich cells are cooled to an average temperature between about −10 and 0° C. for a desired period of time and then cooled to an average temperature between about 0 and 5° C.

In some embodiments, the lipid-rich cells are cooled to an average first temperature range for a first period of time and cooled to an average second temperature range for a second period of time. In some embodiments, the lipid-rich cells are cooled to an average first temperature range between about −10 and 10° C., an average first temperature range between about −10 and 5° C., an average first temperature between about −10 and 0° C., or an average first temperature between about −10 and −5° C. In some embodiments, the lipid-rich cells are cooled to an average second temperature range between about −5 and 15° C., an average second temperature range between about −5 and 10° C., an average second temperature range between about −5 and 5° C., or an average second temperature range between about −5 and 0° C.

In some embodiments, the lipid-rich cells are cooled for a first period of time in short intervals, for example, 1 minute, 5 minute, 15 minute, 30 minute, 60 minute time intervals, or long intervals, for example, 12 hour or 24 hour time intervals. The first period of time is preferably between about 0 and 20 minutes and the second period of time is preferably between about 20 minutes and 80 minutes.

The treatment system 100 can be applied for any duration, for example, 90 minutes. Preferably, the treatment system 100 is applied for between 30 to 90 minutes. The treatment system 100 can be applied at any schedule and any combination of locations on a user's body. For example, a target area can be treated by the treatment system 100 once per day, three days per week, one week a month, for three months. By way of another example, the treatment system 100 is applied twice a day, alternating different locations on a user's body between each daily application, five days a week, two weeks a month, for six months.

The foregoing has described the principles, embodiments, and modes of operation of the present invention. However, the invention should not be construed as being limited to the particular embodiments described above, as they should be regarded as being illustrative and not as restrictive. It should be appreciated that variations may be made in those embodiments by those skilled in the art without departing from the scope of the present invention.

Modifications and variations of the present invention are possible in light of the above teachings. It is therefore to be understood that the invention may be practiced otherwise than as specifically described herein.

What is claimed is:

1. A treatment system for extracting heat from subcutaneous lipid-rich areas, comprising:
an applicator having a cavity, and a retention mechanism, and
a cooling member,
wherein the cavity contains the cooling member, and the retention mechanism secures the applicator to a target area.

2. The treatment system of claim 1 wherein the system is configured to reduce the temperature of a target area such that the subcutaneous lipid-rich area is affected.

3. The treatment system of claim 1 wherein the cavity slidably engages the retention mechanism.

4. The treatment system of claim 1 wherein the retention mechanism comprises a strap, plate, aperture, fastening mechanism, and loop.

5. The treatment system of claim 1 wherein the fastening mechanism secures the treatment system to a location on the user's body.

6. The treatment system of claim 1 wherein the fastening mechanism is adjustable to increase or decrease the pressure applied to a target area.

7. The treatment system of claim 1 wherein the fastening mechanism applies pressure to the target area that is approximately equal to or greater than the systolic blood pressure in the user's non-lipid-rich area of the target area.

8. The treatment system of claim 1 wherein the fastening mechanism is adjustable to allow for the system to be used on a plurality of locations on the user's body.

9. The treatment system of claim 1 further comprising an opening wherein the cooling member passes through the opening.

10. The treatment system of claim 1 wherein the applicator comprises a flexible section for allowing the circumference of the loop to be altered.

11. The treatment system of claim 1 wherein the applicator comprises a closure mechanism.

12. The treatment system of claim 1 wherein the applicator comprises an opening device.

13. The treatment system of claim 1 wherein the applicator comprises a temperature control mechanism.

14. The treatment system of claim 1 wherein the temperature control mechanism is a cryoprotectant.

15. The treatment system of claim 1 wherein the cooling member has an acuate shape for forming to the contour o the target area.

16. The treatment system of claim 1 wherein the cooling member is cooled.

17. A method for affecting lipid-rich cells comprising:
applying a treatment system to a target area to affect a subcutaneous lipid-rich area,
wherein the treatment system has a cooling member and an applicator, and the applicator has a cavity and retention mechanism.

18. The method of claim 17, wherein the cooling member is maintained at an average temperature between about −20 and 10° C.

19. The method of claim 17, wherein the cooling member is maintained at an average temperature between about −20 and 5° C.

20. The method of claim 17, wherein the cooling member is maintained at an average temperature between about −20 and 0° C.

21. The method of claim 17, wherein the cooling member is maintained at an average temperature between about −20 and −5° C.

22. The method of claim 17, wherein the cooling member is maintained at an average temperature between about −20 and −10° C.

23. The method of claim 17, wherein the cooling member is maintained at an average temperature between about −20 and −15° C.

24. The method of claim 17, wherein the lipid-rich cells are cooled to an average temperature between about −10 and 10° C.

25. The method of claim 17, wherein the lipid-rich cells are cooled to an average temperature between about −10 and 5° C.

26. The method of claim 17, wherein the lipid-rich cells are cooled to an average temperature between about −10 and 0° C.

27. The method of claim 17, wherein the lipid-rich cells are cooled to an average temperature between about −10 and −5° C.

28. The method of claim 17, wherein the lipid-rich cells are cooled to an average first temperature range for a first period of time and cooled to an average second temperature range for a second period of time.

29. The method of claim 28, wherein the average first temperature range is between about −5 and 0° C.

30. The method of claim 28, wherein the average second temperature range is between about 0 and 5° C.
